(19) United States
(12) Patent Application Publication
(10) Pub. No.: US 2002/0143065 A1
(43) Pub. Date: Oct. 3, 2002

(54) Developing a delivery system for multi-pharmaceutical active materials at various release rates

(76) Inventors: Lirong Liu, Washington Township, NJ (US); Alex Shlyankevich, Ridgefield, CT (US); Anand Baichwal, Wappingers Falls, NY (US)

Correspondence Address: HALE AND DORR, LLP, 60 STATE STREET, BOSTON, MA, 02109

(21) Appl. No.: 09/970,020

(22) Filed: Oct. 3, 2001

Related U.S. Application Data
(60) Provisional application No. 60/237,451, filed on Oct. 3, 2000.
(60) Provisional application No. 60/239,362, filed on Oct. 11, 2000.

(51) Int. Cl. A61K 31/137; A61K 9/24

(52) U.S. Cl. 514/649; 424/471

(57) ABSTRACT

The invention provides a novel pharmaceutical compositions useful for the delivery of more than one pharmaceutically active compound. More specifically, the invention provides a novel pharmaceutical compositions useful for the delivery of the (+) tramadol enantiomer and the (−) tramadol enantiomer.

### CROSS-REFERENCE TO RELATED APPLICATIONS

This application claims the benefit of U.S. Provisional Application No. 60/237,451, filed Oct. 3, 2000, and U.S. Provisional Application No. 60/239,362, filed Oct. 11, 2000.

### BACKGROUND OF THE INVENTION

1. Field of the Invention

The invention relates to the fields of pharmacology and medicine. More specifically, the invention relates to a pharmaceutical delivery system suitable for use with more than one pharmaceutically active agent, particularly enantiomers of chiral drugs.

2. Description of Related Art

The advantages of controlled release products are well known in the pharmaceutical field and include the ability to maintain a desired blood level of a medicament over a comparatively longer period of time while increasing patient compliance by reducing the number of administrations necessary to achieve the same. These advantages have been attained by a wide variety of methods. For example, different hydrogel have been described for use in controlled release medicines, some of which are synthetic, but most of which are semi-synthetic or of natural origin. A few contain both synthetic and non-synthetic material. However, some of the systems require special process and production equipment, and in addition some of these systems are susceptible to variable drug release.

A reliable controlled release pharmaceutical system is particularly important when it is desirable to include in a single therapy at least two pharmaceutically active agents, which may differ from one another with respect to treatment efficacy, pharmokinetics and toxicity. For example, where the different enantiomers of the chiral drug are absorbed, metabolized, distributed or secreted by the body at different rates, their rates of release from the dosage form may be arranged such that their initial ratio, whether this is 50:50 or a non-racemic ratio, is maintained, ideally throughout the dosing period. By manipulating the administration of the different enantiomers in this way, presentation of the desired enantiomer to the target organ may be optimized, thereby increasing the clinical efficacy of the drug throughout the dosing period.

Examples of chiral drugs where both enantiomers have a separable and valid pharmacological value, and where a clinical benefit may be realized by controlling the release rates of those enantiomers, include warfarin, tramadol, mianserin, carvedilol, citalopram, dobutamine, aminoglutethimide, alfuzosin, celiprolol, cisapride, disopyramide, fenoldopam, flecainide, hydroxychloroquine, ifosfamide, labetolol, mexiletine, propafenone, tegafur, terazosin, thioctic acid, thiopental and zacopride.

By way of example, in regards to the management of pain, the generic drug tramadol is formulated as the racemate for use as a high-potency analgesic with opioid- like properties. Tramadol, a racemate, consists of equal amounts of the (+) and (−) tramadol enantiomers. It is known that the individual tramadol enantiomers have a different pharmaceutical profile from that of the tramadol racemate. The (+) enantiomer is characterized by an opiate-like analgesic effect which is considerably enhanced compared with that of tramadol, while a significant inhibition of noradrenaline reassimilation is observed with the (−) enantiomer.

The analgesic efficacy and safety of the tramadol racemate and the individual tramadol enantiomers have been investigated in a randomized, double-blind study with gynecological patients using intravenous patient-controlled analgesia (see Grond, S. et al., Pain (1995) 62(3):313-320). Although (+) tramadol appeared to be more potent in producing analgesia, it also produced more nausea and vomiting. Since the racemate has more efficacy than (−) tramadol enantiomer and no more side effects than (+) tramadol enantiomer, the authors concluded that the racemate had more clinical utility. In another study, it was shown that there is complementary and synergistic interaction between the individual tramadol enantiomers (see Raffa, R. B. et al., J Pharmacol. Exp. Ther. (1993) 267(1): 331-340). The tramadol enantiomers have different potencies at opioid receptors and in inhibiting serotonin re-uptake and noradrenaline re-uptake. It therefore appears that both enantiomers of tramadol contribute to the analgesic effect. Thus, it is possible that controlled administration of the individual tramadol enantiomers at different rates may result in an even more useful analgesia without additional side effects.

Chiral drugs have previously been administered through the use of controlled release drug delivery systems. However, in some instances, the need or advantage of delivering the separable enantiomers in a controlled fashion has not been recognized. For example, U.S. Pat. No. 5,591,452 by Miller, et al., describes a controlled release drug delivery system for tramadol. The system does not provide for an individualized delivery schedule for the tramadol enantiomers, which differ in pharmacological action and physical properties.

Other controlled-release drug delivery systems recognize the pharmacological importance of enantiomers but fail to provide a system designed for the separate delivery of the two enantiomers. Edgren, et al., (U.S. Pat. Nos. 5,338,550 and 5,204,116) disclose a dosage form comprising a first layer and a second layer. The first layer provides immediate therapy and comprises a drug enantiomer, and the second layer provides prolonged therapy and comprises a drug racemate. Thus, with the system of Edgren, et al., only one enantiomer is administered in an isolated fashion, since the controlled release layer in this system is designed for the delivery of a racemate.

U.S. Pat. No. 6,056,968 by Gilbert, et al. describes a controlled release formulation for the delivery of the (+) tramadol enantiomer and the (−) tramadol enantiomer. However, there is still a need in the art for the development of other controlled release formulations that would provide alternative modes and rates of drug delivery.

The present application provides a novel controlled release delivery system useful for the administration of more than one pharmaceutically efficacious agent. More particularly, the invention provides novel pharmaceutical compositions useful for the delivery of tramadol enantiomers.

### SUMMARY OF THE INVENTION

It has been discovered that novel formulations of TIMERx™ are useful in a drug delivery system designed for the oral administration of more than one pharmaceutically active agent, particularly the administration of enantiomers of chiral compounds useful as pharmaceuticals.

These discoveries have been applied to provide the present invention, which includes pharmaceutical compositions, most particularly pharmaceutical compositions comprising the (+) enantiomer and the (−) enantiomer of tramadol.

In a first aspect, the invention provides pharmaceutical compositions wherein the percentage of TIMERx™-N or TIMERx™-O in the final product is between about 15%-60%.

In a second aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation. As presented in Table 1, when measured by the USP type II dissolution method, the in vitro dissolution rates for the chiral compound enantiomers contained in the CR formulation and the IR formulation are:

TABLE 1
In Vitro CR and IR Dissolution Rates

| Time (hours) | % CR Release | % IR Release |
|---|---|---|
| 0 | 0% | 0% |
| 0.3 | 0-60% | 20-100% |
| 0.5 | 0-65% | 20-100% |
| 1.0 | 5-70% | 25-100% |
| 2.0 | 5-75% | 25-100% |
| 4.0 | 10-80% | 30-100% |
| 6.0 | 10-100% | 30-100% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In a third aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation. As presented in Table 2, when measured by the USP type II dissolution method, the in vitro dissolution rates for the CR formulation and the IR formulation are:

TABLE 2
In Vitro CR and IR Dissolution Rates

| Time (hours) | % CR Release | % IR Release |
|---|---|---|
| 0 | 0% | 0% |
| 0.3 | 0-30% | 0-60% |
| 0.5 | 0-30% | 0-60% |
| 1.0 | 5-70% | 25-70% |
| 2.0 | 5-75% | 25-70% |
| 4.0 | 10-80% | 30-80% |
| 6.0 | 10-100% | 30-80% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In a fourth aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the chiral compound enantiomer, or a pharmaceutically acceptable salt thereof , each chiral compound enantiomer formulated separately, either as an immediate release (IR) formulation or as a controlled release (CR) formulation. As presented in Table 3, when administered to a patient, the pharmaceutical composition provides the following percent of maximum (+) and (−) chiral compound enantiomer plasma concentrations are:

TABLE 3
Percent Maximum Compound Plasma Levels

| Time (hours) | (+) or (−) Enantiomer | (−) or (+) Enantiomer |
|---|---|---|
| 0 | 0% | 0% |
| 0.3 | 0-60% | 0-100% |
| 0.5 | 0-65% | 0-100% |
| 1.0 | 5-70% | 25-100% |
| 2.0 | 5-75% | 25-100% |
| 4.0 | 10-80% | 30-100% |
| 6.0 | 20-100% | 30-100% |
| 8.0 | 20-100% | 20-100% |
| 10.0 | 20-100% | 20-100% |
| 12.0 | 10-100% | 0-90% |
| 18.0 | 0-80% | 0-80% |
| 24.0 | 0-80% | 0-80%. |

In a fifth aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation. As presented in Table 4, when administered to a patient, the pharmaceutical composition provides the following percent maximum (+) and (−) chiral compound enantiomer plasma concentrations:

TABLE 4
Percent Maximum Compound Plasma Levels

| Time (hours) | (+) or (−) Enantiomer | (−) or (+) Enantiomer |
|---|---|---|
| 0 | 0% | 0% |
| 0.3 | 0-40% | 0-100% |
| 0.5 | 0-45% | 0-100% |
| 1.0 | 5-50% | 25-100% |
| 2.0 | 5-55% | 25-100% |
| 4.0 | 10-80% | 30-100% |
| 6.0 | 20-100% | 30-100% |
| 8.0 | 20-100% | 20-100% |
| 10.0 | 10-100% | 20-100% |
| 12.0 | 0-80% | 10-90% |
| 18.0 | 0-80% | 0-80% |
| 24.0 | 0-80% | 0-80%. |

In a sixth aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation.

In a seventh aspect, the invention provides a pharmaceutical composition comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. As presented in Table 5, when measured by the USP type II dissolution method, the in vitro dissolution rates for the CR formulation and the IR formulation are:

TABLE 5
In Vitro Tramadol Enantiomer Dissolution Rate #1

| | % (+) Tramadol | % (−) Tramadol |
|---|---|---|
| Time (hours) | Enantiomer Release | Enantiomer Release |
| 0 | 0% | 0% |
| 0.3 | 0-60% | 20-100% |
| 0.5 | 0-65% | 20-100% |
| 1.0 | 5-70% | 25-100% |
| 2.0 | 5-75% | 25-100% |
| 4.0 | 10-80% | 30-100% |
| 6.0 | 10-100% | 30-100% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In an eighth aspect, the invention provides a pharmaceutical composition comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. As presented in Table 6, when measured by the USP type II dissolution method, the in vitro dissolution rates for the CR formulation and the IR formulation are:

TABLE 6
In Vitro Tramadol Enantiomer Dissolution Rate #2

| | % (+) Tramadol | % (−) Tramadol |
|---|---|---|
| Time (hours) | Enantiomer Release | Enantiomer Release |
| 0 | 0% | 0% |
| 0.3 | 0-30% | 0-60% |
| 0.5 | 0-30% | 0-60% |
| 1.0 | 5-70% | 25-70% |
| 2.0 | 5-75% | 25-70% |
| 4.0 | 10-80% | 30-80% |
| 6.0 | 10-100% | 30-80% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In a ninth aspect, the invention provides a pharmaceutical composition for oral delivery administration comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. As presented in Table 7, when administered to a patient, the pharmaceutical composition provides the following percent of maximum plasma concentrations for the (+) and (−) tramadol enantiomers:

TABLE 7
Percent Maximum Tramadol Enantiomer Plasma Levels

| Time (hours) | (+) Enantiomer | (−) Enantiomer |
|---|---|---|
| 0 | 0% | 0% |
| 0.3 | 0-60% | 0-100% |
| 0.5 | 0-65% | 0-100% |
| 1.0 | 5-70% | 25-100% |
| 2.0 | 5-75% | 25-100% |
| 4.0 | 10-80% | 30-100% |
| 6.0 | 20-100% | 30-100% |
| 8.0 | 20-100% | 20-100% |
| 10.0 | 20-100% | 20-100% |
| 12.0 | 10-100% | 0-90% |
| 18.0 | 0-80% | 0-80% |
| 24.0 | 0-80% | 0-80%. |

In a tenth aspect, the invention provides a pharmaceutical composition for oral delivery administration comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. As presented in Table 8, when administered to a patient, the pharmaceutical composition provides the following percent maximum plasma concentrations for the (+) and (−) tramadol enantiomers:

TABLE 8
Percent Maximum Compound Plasma Levels

| Time (hours) | (+) Enantiomer | (−) Enantiomer |
|---|---|---|
| 0 | 0% | 0% |
| 0.3 | 0-40% | 0-100% |
| 0.5 | 0-45% | 0-100% |
| 1.0 | 5-50% | 25-100% |
| 2.0 | 5-55% | 25-100% |
| 4.0 | 10-80% | 30-100% |
| 6.0 | 20-100% | 30-100% |
| 8.0 | 20-100% | 20-100% |
| 10.0 | 10-100% | 20-100% |
| 12.0 | 0-80% | 10-90% |
| 18.0 | 0-80% | 0-80% |
| 24.0 | 0-80% | 0-80%. |

In an eleventh aspect, the invention provides a pharmaceutical composition comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. In a particularly preferred embodiment, the pharmaceutical composition for oral administration is in the form of a bi-layered tablet.

In a twelfth aspect of the invention, the tramadol-containing formulations of the invention comprise a bi-layer tablet. As presented in Table 9, the tablet consists of a controlled release formulation consisting of:

TABLE 9
Controlled Release Formulation

| Ingredients | A | | (%) |
|---|---|---|---|
| 1. (+) Tramadol HCl | 50 | mg | 5.4 |
| 2. TIMERx ™-N | 350 | mg | 37.7 |
| 3. Proslov | 150 | mg | 16.2 |
| 4. Magnesium Stearate | 5.5 | mg | 0.6 |
| Total | 555.5 | mg | 59.9; |

and

an immediate release formulation consisting of:

TABLE 10
Immediate Release Formulation

| Ingredients | A | | (%) |
|---|---|---|---|
| 1. (−) Tramadol HCl | 150 | mg | 16.2 |
| 2. Prosolv | 100 | mg | 10.8 |
| 3. Lactose Fast-Flow | 100 | mg | 10.8 |
| 4. Explotab | 20 | mg | 2.2 |
| 5. Magnesium Stearate | 3 | mg | 0.3 |
| Total | 373 | mg | 40.3 |

### BRIEF DESCRIPTION OF THE FIGURES

The foregoing and other objects of the present invention, the various features thereof, as well as the invention itself may be more fully understood from the following description, when read together with the accompanying drawings in which:

FIG. 1 is a graphic representation of the dissolution rate of the (+) tramadol enantiomer from formulations in which the percentage of TIMERx™-N is varied.

FIG. 2 is a graphic representation demonstrating the effect of different grades of TIMERx™ (TIMERx™-N versus TIMERx™-O) on the dissolution rate of the (+) tramadol enantiomer.

FIG. 3 is a graphic representation of the in vitro dissolution profile the (+) tramadol enantiomer and the (−) tramadol enantiomers contained in a controlled release and an immediate release layer of a bi-layer tablet, respectively.

FIG. 4 is a graphic representation of the mean plasma profile for the (+) tramadol enantiomer and the (−) tramadol enantiomer contained in a controlled release formulation and an immediate release formulation of a bi-layer tablet, respectively.

### DETAILED DESCRIPTION OF THE PREFERRED EMBODIMENTS

The patent and scientific literature cited herein establishes the knowledge that is available to those with skill in the art. The issued U.S. patents, allowed applications, published foreign applications, and references cited herein are hereby incorporated by reference.

The invention herein utilizes novel formulations of TIMERx™, formulations referred to as TIMERx™-N and TIMERx™-O in the production of novel pharmaceutical compositions. TIMERx™ was previously described in, for example, in U.S. Pat. Nos. 6,048,548; 5,962,009; 5,958,456; and 5,846,563.

The pharmaceutical formulations provided by the invention are useful for the administration of compounds with a water solubility range of less than 10⁻⁶ grams per milliliter (relatively insoluble) to more than 100 grams per milliliter (very soluble). The pharmaceutical formulations of the invention are particularly suited for the administration of compounds soluble in water.

In a first aspect, the invention provides pharmaceutical compositions wherein the percentage of TIMERx™-N or TIMERx™-O in the final formulation is between about 15%-60%. In a preferred embodiment thereof, the percentage of TIMERx™-N or TIMERx™-O in the final product is between about 25-50%. In a more preferred embodiment thereof, the percentage of TIMERx™-N or TIMERx™-O in the final product is between about 35%-45%. In a most preferred embodiment thereof, the percentage of TIMERx™-N or TIMERx™-O in the final product is 38%.

The term “chirality of a drug” is used herein to denote that the drug exists in alternative molecular forms, referred to by the term “stereoisomers” or “enantiomers.” Enantiomers are distinguished in one way by their ability to rotate the plane of polarized light. One enantiomer rotates the plane of light to the right, (called dextrorotatory, d or +), while the other enantiomer rotates the plane of light to the left, (levorotatory, 1 or −). A racemic mixture comprises an equal number of (+) and (−) stereoisomer molecules. The racemic mixture is essentially free of optical activity.

By “substantially single enantiomer” typically is meant that one enantiomer is in an excess of at least 70% by weight with respect to the other enantiomer, and is preferably in an excess of at least 80%, and more preferably 90%, or higher. Furthermore, by a “non-racemic ratio of enantiomers” typically is meant that both enantiomers are present, with either the (−) enantiomer being present in an amount in excess of that of the (+) enantiomer, or vice versa.

By “controlled release” it is meant for purposes of the present invention that the therapeutically active medicament is released from the formulation at a controlled rate such that therapeutically beneficial blood levels (but below toxic levels) of the medicament are maintained over an extended period of time, e.g., providing a dosage form which provides effective levels of the medicament in vivo for a time period of from about 1 to about 24 hours or more.

A number of release profiles for the different enantiomers of a chiral drug may be realized by way of the dosage forms of the present invention. For instance, a dosage form may be designed to allow immediate release of one enantiomer and sustained, or controlled, release of the other enantiomer. In this case, by “immediate release” typically is meant that release of the respective enantiomer occurs substantially immediately or after only a short delay, usually no more than five to ten minutes, after administration of the dosage form, and continues usually over a period of up to one to two hours. By “sustained release” or “controlled release” typically is meant that release of the respective enantiomer is delayed usually for at least one hour and frequently longer, for instance for two or more hours, after administration of the dosage form. The sustained release or controlled release may be constant or variable throughout the treatment period.

The dosage forms of the present invention may be designed to release either of the enantiomers faster than the other, or before the other, depending upon the condition to be treated, or the patient type. It may be desirable to maintain a constant ratio of the separate enantiomers at the target tissue over a specified period of time, for instance at least 8 hours a day, preferably at least 12 hours a day, most preferably 24 hours a day. The ratio maintained may be 50:50, or a non-racemic ratio in which either the amount of the (+) enantiomer is greater than the (−) enantiomer, or vice versa.

Another option is to vary the ratio of the two enantiomers throughout the treatment period, or at least for a portion of that period. For instance, the release rate of either or both enantiomers can be arranged to vary, so that either the relative proportion of the (+) enantiomer or of the (−) enantiomer increases, or decreases, with time. The latter may be achieved, for instance, by using a number of different release coatings for the respective enantiomer.

The controlled release solid dosage form can be prepared in any conventional orally administered dosage form, including a tablet, as a granular form and as a granular form administered in a gelatin capsule containing a sufficient amount of the granules to provide an effective dose of the included therapeutically active medicament. For a tablet dosage form, at least part of a surface of the tablet can optionally be coated with a hydrophobic material to a weight gain from about 1 to about 20%, by weight. Further, a granular dosage form can optionally be coated with a hydrophobic coating material to a weight gain that ranges from about 1% to about 20%. The hydrophobic material can be selected from, e.g., a cellulose ether, a cellulose ester and an alkylcellulose. The hydrophobic material can optionally be applied before, during or after the process of creating the tablet. In addition, if there is a need for an early release of the active medicament, the coating can optionally be formulated to include from about 10 to about 40% of the total amount of the active medicament in a quick release external layer In a second aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation. When measured by the USP type II dissolution method, the in vitro dissolution rates for the chiral compound enantiomers contained in the CR formulation and the IR formulation are:

TABLE 1
In Vitro CR and IR Dissolution Rates

| Time (hours) | % CR Release | % IR Release |
|---|---|---|
| 0 | 0% | 0% |
| 0.3 | 0-60% | 20-100% |
| 0.5 | 0-65% | 20-100% |
| 1.0 | 5-70% | 25-100% |
| 2.0 | 5-75% | 25-100% |
| 4.0 | 10-80% | 30-100% |
| 6.0 | 10-100% | 30-100% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In a third aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation. When measured by the USP type II dissolution method, the in vitro dissolution rates for the CR formulation and the IR formulation are:

TABLE 2
In Vitro CR and IR Dissolution Rates

| Time (hours) | % CR Release | % IR Release |
|---|---|---|
| 0 | 0% | 0% |
| 0.3 | 0-30% | 0-60% |
| 0.5 | 0-30% | 0-60% |
| 1.0 | 5-70% | 25-70% |
| 2.0 | 5-75% | 25-70% |
| 4.0 | 10-80% | 30-80% |
| 6.0 | 10-100% | 30-80% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In a particularly preferred embodiment, the pharmaceutical composition for oral administration is in the form of a bi-layered tablet. In an especially preferred embodiment, the pharmaceutical composition comprising tramadol (+) and (−) enantiomers includes formulations designed to provide appropriate administration to a patient without the undesirable known side effects attributed to one or the other enantiomer.

The term “about” is used herein to mean “approximately,” or “roughly,” or “around,” or “in the region of.” When the term “about” is used in conjunction with a numerical range, it modifies that range by extending the boundaries above and below the numerical values set forth. In general, the term “about” is used herein to modify a numerical value above and below the stated value by a variance of 20%. Thus, if it is stated that about 20% of the pharmaceutically active compound, e.g., an enantiomer, is released after one hour of administration, it is understood that approximately 18%-22% of the compound is released after one hour of administration.

The term “percent” as used herein refers to a weight/weight value unless otherwise indicated.

In other embodiments, the pharmaceutical compositions of the invention comprising (+) and (−) chiral compound enantiomers includes a controlled release (CR) formulation that further comprises TIMERx™-N and one chiral compound enantiomer such that a gum to drug ratio of between about 1:3 to 3:1, respectively, is established. Alternatively, other embodiments of the pharmaceutical compositions of the invention include a CR formulation that further comprises TIMERx™-O and one chiral compound enantiomer such that a gum to drug ratio of between about 1:3 to 3:1, respectively, is established.

Various embodiments of the invention are drawn to formulations in which the (+) chiral compound enantiomer and the (−) chiral compound enantiomer are present in the composition at different mass quantities. The invention includes any and all formulations varying the ratio of one enantiomer to the other found to be clinically effective for the desired treatment. In a preferred embodiment, the percentage of each enantiomer present in the formulations will vary from one another at a ratio of the (+) chiral compound enantiomer and the (−) chiral compound enantiomer selected from a 2:1, or a 3:1, or a 4:1, or a 5:1, or a 10:1, or a 1:2, or a 1:3, or a 1:4, or a 1:5, or a 1:10 ratio, respectively.

In a preferred embodiment, the pharmaceutical formulation is designed so that about 80%, or about 90%, or about 95%, or about 100% of the (+) chiral compound enantiomer and about 80%, or about 90%, or about 95%, or about 100% of the (−) chiral compound enantiomer are released within about 12 hours of administration.

In a fourth aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation. When administered to a patient, the pharmaceutical composition provides the following percent of maximum (+) and (−) chiral compound enantiomer plasma concentrations are:

TABLE 3
Percent Maximum Compound Plasma Levels

| | (+) or (−) | (−) or (+) |
|---|---|---|
| Time (hours) | Enantiomer | Enantiomer |
| 0 | 0% | 0% |
| 0.3 | 0-60% | 0-100% |
| 0.5 | 0-65% | 0-100% |
| 1.0 | 5-70% | 25-100% |
| 2.0 | 5-75% | 25-100% |
| 4.0 | 10-80% | 30-100% |
| 6.0 | 20-100% | 30-100% |
| 8.0 | 20-100% | 20-100% |
| 10.0 | 20-100% | 20-100% |
| 12.0 | 10-100% | 0-90% |
| 18.0 | 0-80% | 0-80% |
| 24.0 | 0-80% | 0-80%. |

In a fifth aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation. When administered to a patient, the pharmaceutical composition provides the following percent of maximum (+) and (−) chiral compound enantiomer plasma concentrations are:

TABLE 4
Percent Maximum Compound Plasma Levels

| | (+) or (−) | (−) or (+) |
|---|---|---|
| Time (hours) | Enantiomer | Enantiomer |
| 0 | 0% | 0% |
| 0.3 | 0-40% | 0-100% |
| 0.5 | 0-45% | 0-100% |
| 1.0 | 5-50% | 25-100% |
| 2.0 | 5-55% | 25-100% |
| 4.0 | 10-80% | 30-100% |
| 6.0 | 20-100% | 30-100% |
| 8.0 | 20-100% | 20-100% |
| 10.0 | 10-100% | 20-100% |
| 12.0 | 0-80% | 10-90% |
| 18.0 | 0-80% | 0-80% |
| 24.0 | 0-80% | 0-80%. |

In a sixth aspect, the invention provides a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the chiral compound enantiomer, or a pharmaceutically acceptable salt thereof, and the (+) chiral compound enantiomer, or a pharmaceutically acceptable salt thereof, each chiral compound enantiomer formulated separately, either as an immediate release (IR) formulation or as a controlled release (CR) formulation.

Examples of chiral drugs where both enantiomers have a separable and valid pharmacological value, and where a clinical benefit may be realized by controlling the release rates of those enantiomers, include warfarin, tramadol, mianserin, carvedilol, citalopram, dobutamine, aminoglutethimide, alfuzosin, celiprolol, cisapride, disopyramide, fenoldopam, flecainide, hydroxychloroquine, ifosfamide, labetolol, mexiletine, propafenone, tegafur, terazosin, thioctic acid, thiopental and zacopride.

In particularly preferred aspect of the invention, the formulations described herein are useful for the administration of tramadol, particularly the (+) enantiomer and the (−) enantiomer thereof. The enantiomers of tramadol may be conveniently isolated in relatively pure form using known methods in the art, for example as described in U.S. Pat. No. 5,723,668, which provides a method of separating the racemate of tramadol.

Thus, a seventh aspect of the invention provides a pharmaceutical composition comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. When measured by the USP type II dissolution method, the in vitro dissolution rates for the CR formulation and the IR formulation are:

TABLE 5
In Vitro Tramadol Enantiomer Dissolution Rate #1

| | % (+) Tramadol | % (−) Tramadol |
|---|---|---|
| Time (hours) | Enantiomer Release | Enantiomer Release |
| 0 | 0% | 0% |
| 0.3 | 0-60% | 20-100% |
| 0.5 | 0-65% | 20-100% |
| 1.0 | 5-70% | 25-100% |
| 2.0 | 5-75% | 25-100% |
| 4.0 | 10-80% | 30-100% |
| 6.0 | 10-100% | 30-100% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In eighth aspect, the invention provides a pharmaceutical composition comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. When measured by the USP type II dissolution method, the in vitro dissolution rates for the CR formulation and the IR formulation are:

TABLE 6
In Vitro Tramadol Enantiomer Dissolution Rate #2

| | % (+) Tramadol | % (−) Tramadol |
|---|---|---|
| Time (hours) | Enantiomer Release | Enantiomer Release |
| 0 | 0% | 0% |
| 0.3 | 0-30% | 0-60% |
| 0.5 | 0-30% | 0-60% |
| 1.0 | 5-70% | 25-70% |
| 2.0 | 5-75% | 25-70% |
| 4.0 | 10-80% | 30-80% |
| 6.0 | 10-100% | 30-80% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

In a particularly preferred embodiment, the pharmaceutical composition for oral administration is in the form of a bi-layered tablet. In an especially preferred embodiment, the pharmaceutical composition comprising tramadol (+) and (−) enantiomers includes formulations designed to provide appropriate administration to a patient without the undesirable known side effects attributed to one or the other enantiomer.

In other embodiments, the pharmaceutical compositions of the invention comprising tramadol (+) and (−) enantiomers includes a controlled release (CR) formulation that further comprises TIMERx™-N and one tramadol enantiomer such that a gum to drug ratio of between about 1:3 to 3:1, respectively, is established. Alternatively, other embodiments of the pharmaceutical compositions of the invention include a CR formulation that further comprises TIMERx™-O and one tramadol enantiomer such that a gum to drug ratio of between about 1:3 to 3:1, respectively, is established.

Various embodiments of the invention are drawn to formulations in which the (+) tramadol enantiomer and the (−) tramadol enantiomer are present in the composition at different mass quantities. The invention includes any and all formulations varying the ratio of one enantiomer to the other found to be clinically effective for the desired treatment. In a preferred embodiment, the percentage of each enantiomer present in the formulations will vary from one another at a ratio of the (+) tramadol enantiomer and the (−) tramadol enantiomer selected from a 2:1, or a 3:1, or a 4:1, or a 5:1, or a 10:1, or a 1:2, or a 1:3, or a 1:4, or a 1:5, or a 1:10 ratio, respectively.

In a preferred embodiment, the pharmaceutical formulation is designed so that about 80%, or about 90%, or about 95%, or about 100% of the (+) tramadol enantiomer and about 80%, or about 90%, or about 95%, or about 100% of the (−) tramadol enantiomer are released within about 12 hours of administration.

In a ninth aspect, the invention provides a pharmaceutical composition for oral delivery administration comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. When administered to a patient, the pharmaceutical composition provides the following percent of maximum plasma concentrations for the (+) and (−) tramadol enantiomers are:

TABLE 7
Percent Maximum Tramadol Enantiomer Plasma Levels

| Time (hours) | (+) Enantiomer | (−) Enantiomer |
|---|---|---|
| 0 | 0% | 0% |
| 0.3 | 0-60% | 0-100% |
| 0.5 | 0-65% | 0-100% |
| 1.0 | 5-70% | 25-100% |
| 2.0 | 5-75% | 25-100% |
| 4.0 | 10-80% | 30-100% |
| 6.0 | 20-100% | 30-100% |
| 8.0 | 20-100% | 20-100% |
| 10.0 | 20-100% | 20-100% |
| 12.0 | 10-100% | 0-90% |
| 18.0 | 0-80% | 0-80% |
| 24.0 | 0-80% | 0-80%. |

In a tenth aspect, the invention provides a pharmaceutical composition for oral delivery administration comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. When administered to a patient, the pharmaceutical composition provides the following percent of maximum plasma concentrations for the (+) and (−) tramadol enantiomers are:

TABLE 8
Percent Maximum Compound Plasma Levels

| Time (hours) | (+) Enantiomer | (−) Enantiomer |
|---|---|---|
| 0 | 0% | 0% |
| 0.3 | 0-40% | 0-100% |
| 0.5 | 0-45% | 0-100% |
| 1.0 | 5-50% | 25-100% |
| 2.0 | 5-55% | 25-100% |
| 4.0 | 10-80% | 30-100% |
| 6.0 | 20-100% | 30-100% |
| 8.0 | 20-100% | 20-100% |
| 10.0 | 10-100% | 20-100% |
| 12.0 | 0-80% | 10-90% |
| 18.0 | 0-80% | 0-80% |
| 24.0 | 0-80% | 0-80%. |

In an eleventh aspect, the invention provides a pharmaceutical composition comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration. In a particularly preferred embodiment, the pharmaceutical composition for oral administration is in the form of a bi-layered tablet.

In a twelfth aspect of the invention, the tramadol-containing formulations of the invention comprise a bi-layer tablet. The tablet consists of a controlled release formulation consisting of:

TABLE 9
Controlled Release Formulation

| Ingredients | A | | (%) |
|---|---|---|---|
| 1. (+) Tramadol HCl | 50 | mg | 5.4 |
| 2. TIMERx ™-N | 350 | mg | 37.7 |
| 3. Proslov | 150 | mg | 16.2 |
| 4. Magnesium Stearate | 5.5 | mg | 0.6 |
| Total | 555.5 | mg | 59.9; |

and an immediate release formulation consisting of:

TABLE 10
Immediate Release Formulation

| Ingredients | A | | (%) |
|---|---|---|---|
| 1. (−) Tramadol HCl | 150 | mg | 16.2 |
| 2. Prosolv | 100 | mg | 10.8 |
| 3. Lactose Fast-Flow | 100 | mg | 10.8 |
| 4. Explotab | 20 | mg | 2.2 |
| 5. Magnesium Stearate | 3 | mg | 0.3 |
| Total | 373 | mg | 40.3 |

The following examples illustrate the preferred modes of making and practicing the present invention but are not meant to limit the scope of the invention since alternative methods may be utilized to obtain similar results.

### EXAMPLES

### Example 1

### Development of a Delivery System for Multi-Pharmaceutical Active Agents at Various Release-Rates by Using Bi-Layer Tablets

A) Varying the Amount of TIMERx™-N in Controlled Release (CR) Formulations

In order to determine the optimum formulation for the (+) tramadol enantiomer to be delivered in a controlled release fashion, various formulations were tested for efficacy in obtaining the desired controlled release.

As presented in Table 11, a various formulations were tested in a pilot study.

TABLE 11
Formulation (mg per layer) Testing for the Controlled Release of (+)
Tramadol HCl

| Ingredients | A | (%) | B | (%) | C | (%) | D | (%) |
|---|---|---|---|---|---|---|---|---|
| 1. (+) | 50 | 16.6 | 50 | 14.2 | 50 | 12.5 | 50 | 11.1 |
| Tramadol HCl | | | | | | | | |
| 2. | 50 | 16.6 | 100 | 28.5 | 150 | 37.4 | 200 | 44.3 |
| TIMERx ™-N | | | | | | | | |
| 3. Proslov | 200 | 66.5 | 200 | 57.0 | 200 | 49.9 | 200 | 44.3 |
| 4. Magnesium | 1 | 0.3 | 1 | 0.3 | 1 | 0.2 | 1 | 0.2 |
| Stearate | | | | | | | | |
| Total | 301 | 100 | 351 | 100 | 401 | 100 | 451 | 99 |

The following formulation of an Immediate Release (IR) layer (Table 12) was used in this pilot study for the purpose of calculating the total amount of percent polymers that would be contained in a bi-layer tablet.

TABLE 12
Formulation of the Immediate Release Layer (mg per tablet)

| Ingredients | B | (%) |
|---|---|---|
| 1. (−) Tramadol HCl | 200 | 47.4 |
| 2. Prosolv | 100 | 23.7 |
| 3. Lactose Fast-Flow | 100 | 23.7 |
| 4. Explotab | 20 | 4.7 |
| 5. Magnesium Stearate | 2.2 | 0.5 |
| Total | 422.2 | 100 |

The formulations varied on the basis of the amount of TIMERx™-N included in each test formulation. TIMERx™-N is a controlled release polymer system which consists of an insoluble or soluble diluent dispersed in a matrix of a hydrophilic hydratable high polymers such as hydrophilic polysaccharides, hydrocolloids or proteinaceous materials. In the instant study, TIMERx™-N consisted of 25% locust bean gum, 25% xanthan gum, 35% dextrose, 10% calcium sulfate and 5% ethylcellulose. TIMERx™-N was manufactured by wet granulation into a free-flowing polymeric system.

The percent of polymers per controlled release layer ranged from 8.3% to 22.15%, based on the total percent of polymers used (locust bean gum and xanthan gum), which in TIMERx™-N account for 50% of the whole composition.

The percent of polymers per tablet, including the IR and CR layers ranged from 3.45% to 11.45%. These results demonstrated that by varying the percent of polymers in the overall formulation, it is possible to have a different drug release rate.

Standard equipment was utilized for the production of the different formulations to be tested. The equipment included balances, a high-shear mixer, a fluid-bed dryer, (aeromatic STREA-1), a Fitz-Patrick mill, a Patterson Kelly blender and a rotary press, Korsch PH106.

Briefly, in order to produce the controlled release formulation, all ingredients were accurately weighed prior to mixing. Ingredients 1-3 of Table 11 were transferred into high-shear mixer and mixed for 1 minute. Afterwards, the mixture was granulated by adding water into the chamber until a desired granules formed. The granules were then dried in a fluid-bed dryer until loss on drying (LOD) reached less than 5%.

In the next phase of synthesizing the various formulations, ingredient number 4 of Table 11 was blended with the milled granules. The mixture was then compressed into tablet form.

The test controlled release formulations were evaluated by an in vitro dissolution study. The dissolution study is a Type 2 study. Briefly, conditions for the in vitro dissolution analysis were the same as for the USP type II dissolution method. The analysis was done using a Van Kel 8000 Dissolution Sampling Station at a speed of 50 RPM, a volume of 900 ml, a 35 μm flow filter, and a bath temp of 37.0° C.±0.5° C. Sampling station conditions were as follows: a sample volume of 1 ml, a prime time of 90 seconds, a purge time of 90 seconds, Q.C. time points taken at 1, 6, and 12 hours, and time points for evaluation of the release profile were ¼, ½, 1, 2, 4, 6, 8, 10, 12, and 18 hours. A 35 μm flow filter was utilized.

Dissolution test samples were analyzed by high performance liquid chromatography analysis. Briefly, the guard column was a Phenomenex C18 4 mm L×3.0 mm ID, or equivalent. and the analytical column was a Astec Cyclobond I 2000 β-cyclodextrin chiral HPLC column, Cat. No. 20724, 250 mm×4.6 mm. The mobile phase was run with 0.1% TEA, pH 5.0:Acetonitrile:THF (85:15:0.1) v/v/v. The column temperature was 30° C., the injection volume was 20 μL, and the flow Rate 1.0 ml/minute Samples were analyzed at a wavelength of 275 nm. The total run time was about 20 min.

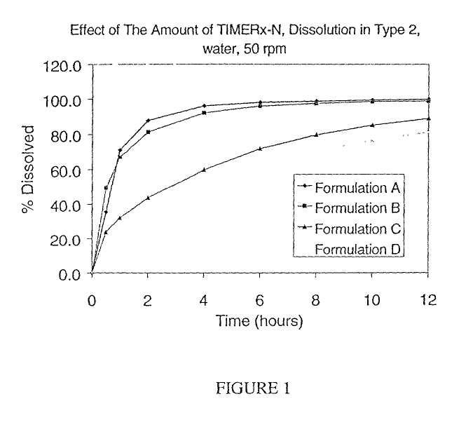

In vitro dissolution results are presented in FIG. 1 and summarized in Table 13.

TABLE 13
Percent of Release of Tramadol In Vitro

| | Formulation | Formulation | Formulation | Formulation |
|---|---|---|---|---|
| Time (hr) | A | B | C | D |
| 0 | 0.0 | 0.0 | 0.0 | 0.0 |
| 0.5 | 35.6 | 49.2 | 23.7 | 17.2 |
| 1 | 71.1 | 67.0 | 32.2 | 25.5 |
| 2 | 87.9 | 81.3 | 43.6 | 36.6 |
| 4 | 96.1 | 92.1 | 59.6 | 51.7 |
| 6 | 98.2 | 96.0 | 71.7 | 62.6 |
| 8 | 98.8 | 97.5 | 79.5 | 70.4 |
| 10 | 99.3 | 98.4 | 84.9 | 76.4 |
| 12 | 99.6 | 98.6 | 88.6 | 80.8 |

In conclusion, the percent of polymers per tablet which includes the immediate release and the controlled release layers ranged from 3.45% to 11.45%. As the amount of TIMERx™-N in controlled release formulation increased, the in vitro release rate decreased. These results indicated that by varying the amount of TIMERx™-N in the formulation, the desired controlled release profile could be obtained.

B) The Effect of TIMERx™-N and TIMERx™-O on the Controlled Release

In order to examine the effect of varying the grade of TIMERx™, e.g., TIMERx™-N and TIMERx™-O, in controlled release formulations, a pilot study was undertaken. The pharmaceutical formulations tested are indicated in Table 14.

TABLE 14
Formulations Varying the Grade of TIMERx ™ (mg per layer)

| Ingredients | A | (%) | B | (%) |
|---|---|---|---|---|
| 1. (+) Tramadol HCl | 50 | 11.1 | 50 | 11.1 |
| 2. TIMERx ™-N | 200 | 44.3 | — | — |
| 3. TIMERx ™-O | — | — | 200 | 44.3 |
| 4. Proslov | 200 | 44.3 | 200 | 44.3 |
| 5. Magnesium Stearate | 1 | 0.2 | 1 | 0.2 |
| Total | 451 | 99.9 | 451 | 99.9 |

In this study, TIMERx™-N consisted of 25% locust bean gum, 25% xanthan gum, 35% dextrose, 10% calcium sulfate and 5% ethylcellulose and was manufactured by wet granulation into a free-flowing polymeric system. TIMERx™-O consisted of 15% locust bean gum, 15% xanthan gum, 60% dextrose and 10% calcium sulfate and was also manufactured by wet granulation into a free-flowing polymeric system.

Standard equipment was utilized for the production of the different formulations to be tested. The equipment included balances, a high-shear mixer, a fluid-bed dryer (aeromatic STREA-1), a Fitz-Patrick mill, a Patterson Kelly blender and a rotary press, Korsch PH106.

Briefly, (+) tramadol HCl, prosolv, TIMERx™-N (Formulation A) or TIMERx™-O (Formulation B) were transferred into a high-shear mixer and mixed for 1 minute The sample was then granulated by adding water into the chamber until the desired granules formed. The granules were dried in a fluid-bed dryer until loss on drying (LOD) reached less than 5%. Next, magnesium stearate was blended with the milled granules. Afterwards, the material was compressed into tablets using 5/16″ tooling.

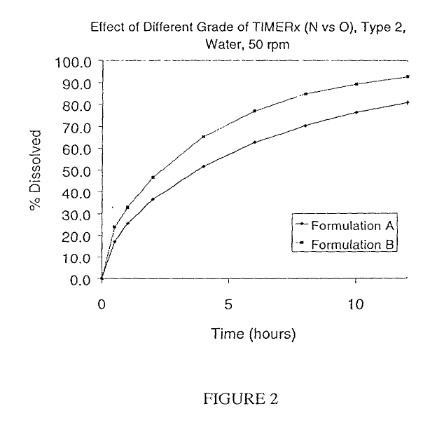

These formulations were then evaluated by the in vitro dissolution assay as previously described. The results are presented graphically in FIG. 2 and presented quantitatively in Table 15.

TABLE 15
Percent Release of Tramadol from
Formulations Varying by TIMERx ™ Grade

| | Formulation | Formulation |
|---|---|---|
| Time (hr) | A | B |
| 0 | 0.0 | 0 |
| 0.5 | 17.2 | 23.8 |
| 1 | 25.5 | 32.9 |
| 2 | 36.6 | 46.7 |
| 4 | 51.7 | 65.3 |
| 6 | 62.6 | 77 |
| 8 | 70.4 | 84.9 |
| 10 | 76.4 | 89.3 |
| 12 | 80.8 | 92.6 |

In this study, TIMERx™-N consisted of 50% of gums and other ingredients while TIMERx™-O consisted of 30% of gums and other ingredients. This difference in composition created two distinct release rates when these two different TIMERx™'S were incorporated into a controlled release formulation while maintaining the other ingredients at the same relative percent.

### Example 2

### Clinical Studies Examining Drug Delivery

A) Bi-Layer Tablet Formulation and Manufacture

For the clinical studies described herein, the following formulations for the CR and IR layers of a bi-layered tablet were utilized.

Table 6 presents the CR Layer Formulation used in this study.

TABLE 16
CR Formulation (mg per layer)

| Ingredients | A | (%) |
|---|---|---|
| 1. (+) Tramadol HCl | 50 | 9.0 |
| 2. TTMERx ™-N | 350 | 63.0 |
| 3. Proslov | 150 | 27.0 |
| 4. Magnesium Stearate | 5.5 | 1.0 |
| Total | 555.5 | 100.0 |

Standard equipment was utilized for the production of the formulation to be tested. The equipment included balances, a high-shear mixer (Niro-Fielder PMA25), a fluid-bed dryer (Aeromatic MP-1), a Fitz-Patrick mill, a Patterson Kelly blender and a rotary press, Natoli Type BB.

After carefully weighing all materials, ingredients 1-3 of Table 16 were transferred into a high-shear mixer and mixed for 3 minutes. Granulation was performed by adding water into the chamber until the desired granules formed. The granules were dried in a fluid-bed dryer until loss on drying (LOD) reached less than 5%. Next, the granules were milled through a Fitz-Patrick mill with 0.005″ screen. Finally, the mixture was blended the magnesium stearate with the milled granules for 3 minutes.

The CR granules were used as the lower layer when later compressed into a bi-layer tablet form.

Table 17 presents the IR Layer Formulation used in this study.

TABLE 17
IR Layer Formulation (mg per layer)

| Ingredients | A | (%) |
|---|---|---|
| 1. (−) Tramadol HCl | 150 | 40.2 |
| 2. Prosolv | 100 | 26.8 |
| 3. Lactose Fast-Flow | 100 | 26.8 |
| 4. Explotab | 20 | 5.4 |
| 5. Magnesium Stearate | 3 | 0.8 |
| Total | 373 | 100 |

The following pieces of equipment were utilized in creating the formulation: a high-shear mixer (a Niro-Fielder PMA25), a fluid-bed dryer (Aeromatic MP-l), a Fitz-Patrick mill, a Patterson Kelly Blender and a rotary press (Natoli Type BB).

The tablets were produced in the following fashion. Briefly, after all ingredients were accurately measured, ingredients 1 to 3 of Table 7 were transferred into high-shear mixer and mixed for 3 minutes. Next, the sample was granulated by adding water into the chamber until a desired granules formed. The granules were dried in a fluid-bed dryer until loss on drying (LOD) reached less than 5%. Afterwards, the granules were milled through a Fitz-Patrick mill, and the milled granules later blended with ingredients 4and 5.

The IR granules were used as the upper layer in the compression of the material into a bi-layer tablet form.

The final bi-layered tablet had the following formulation. The formulation of the CR Layer is presented in Table 18.

TABLE 18
CR Layer Formulation (mg/% per tablet)

| Ingredients | A | (%) |
|---|---|---|
| 1. (+) Tramadol HCl | 50 | 5.4 |
| 2. TIMERx ™-N | 350 | 37.7 |
| 3. Proslov | 150 | 16.2 |
| 4. Magnesium Stearate | 5.5 | 0.6 |
| Total | 555.5 | 59.9 |

The formulation of the IR Layer is presented below in Table 19.

TABLE 19
IR Layer Formulation:

| Ingredients | A | (%) |
|---|---|---|
| 1. (−) Tramadol HCl | 150 mg | 16.2 |
| 2. Prosolv | 100 mg | 10.8 |
| 3. Lactose Fast-Flow | 100 mg | 10.8 |
| 4. Explotab | 20 mg | 2.2 |
| 5. Magnesium Stearate | 3 mg | 0.3 |
| Total | 373 mg | 40.3 |
| Overall Tablet Weight (mg) | 928.5 mg | 100.2 |

For this study, TIMERx™-N consisted of 25% locust bean gum, 25% xanthan gum, 35% dextrose, 10 % calcium sulfate and 5% ethylcellulose. TIMERx™-N was manufactured by wet granulation into a free-flowing polymeric system.

The percent of polymers per tablet which gave the desired release profile and which included both the IR and CR layers was 18.9%.

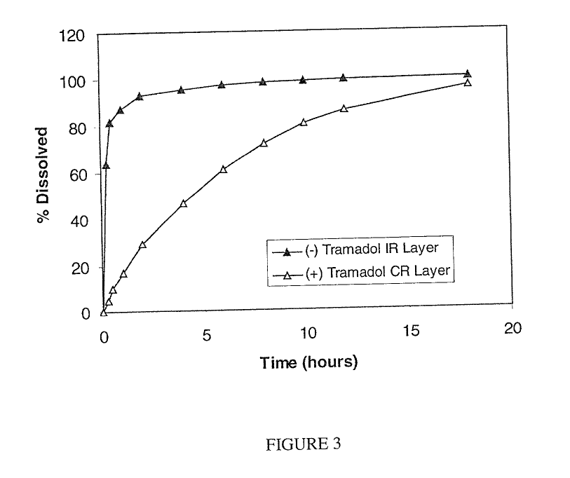

This bi-layer tablet formulation was then evaluated by the in vitro dissolution assay as previously decried. The results are presented graphically in FIG. 3 and presented quantitatively in Table 20 below.

TABLE 20
Dissolution Rate of Tramadol From Bi-layer Tablet

| | (−) Tramadol | (+) Tramadol |
|---|---|---|
| Time (hours) | IR Layer | CR Layer |
| 0.0 | 0 | 0 |
| 0.25 | 64 | 4.7 |
| 0.5 | 81.6 | 10 |
| 1.0 | 87.5 | 16.9 |
| 2.0 | 93 | 29.1 |
| 4.0 | 95.5 | 47 |
| 6.0 | 97.4 | 61.1 |
| 8.0 | 98.2 | 71.8 |
| 10.0 | 98.7 | 80.3 |
| 12.0 | 99.4 | 85.8 |
| 18.0 | 99.9 | 95.9 |

In conclusion, the delivery system based on a bi-layer tablet design did provide for a distinct release rate for each of two pharmaceutically active materials. The (+) tramadol HCl and (−) tramadol HCl were used as the model drugs for this study. The (+) tramadol was formulated for a long controlled release profile, and the (−) tramadol enantiomer was formulated for a rapid or immediate release profile. As shown in above graph, the in vitro release profile indicated that (+) tramadol and (−) tramadol each gave its designed release profile and their profiles were distinctly different from each other.

B) In Vivo Efficacy of the Bi-Layer Tablet Design

The bi-layer tablets based on this delivery system were manufactured and were administered orally to 8 human subjects.

The mean plasma profile for tramadol enantiomer delivery was determined by The concentration of the (+) and (−) tramadol HCl enantiomers in plasma were determined by the LC-MS/MS method.

Briefly, LC-MS/MS conditions are as follows. The flow rate is 2.0 ml per minute. The mobile phase and gradient are as follows:

| | | Gradient | % n-hexane | % ethanol |
|---|---|---|---|---|
| Time | Duration | Profile | (0.2% DEA) | (0.2% DEA) |
| 0 | 8 | 3 | 95 | 5 |
| 8 | 2.2 | 0 | 95 | 5 |
| 10.2 | 0.5 | 1 | 98 | 2 |
| 10.7 | 1.3 | 0 | 98 | 2 |

The autosampler wash was 95:5 n-hexane:ethanol overall 0.2% DEA. The injection volume was 40 microliters and the sheath gas was 13 nitrogen. The mass transitions were as follows:

| | | |
|---|---|---|
| CH5408 and CH5409 | 263.9-58.0 | Dwell Time = 800 ms |
| CH8702 and CH8703 | 250.1-58.0 | Dwell Time = 800 ms |
| Venlafaxine | 278.0-58.0 | Dwell Time = 800 ms |

The collision gas was 3(Nitrogen).

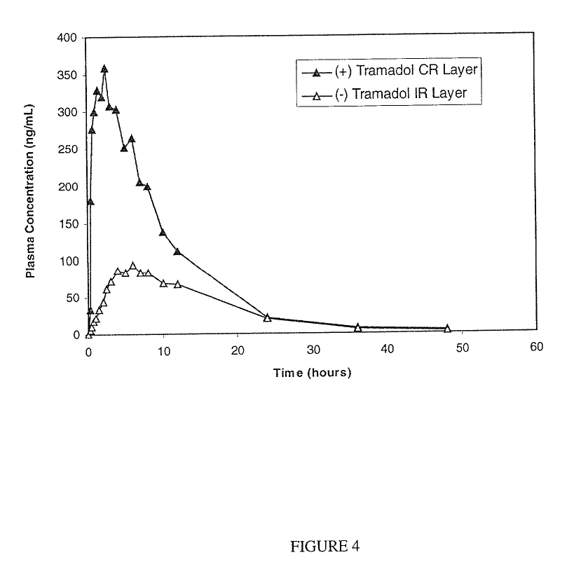

The mean plasma profile for tramadol enantiomer delivery in the patients of this study is presented in FIG. 4 and Table 21. Table 22 presents the plasma concentration maximum (Cmax) and the Area Under the Curve (AUC) for the 48 hour time point of another study.

TABLE 21
Blood Plasma Levels of Tramadol in Enantiomers

| Time | (+) Tramadol | (−) Tramadol |
|---|---|---|
| (hours) | CR Layer | IR Layer |
| 0.00 | 0.0 | 0.0 |
| 0.25 | 33.9 | 5.2 |
| 0.50 | 181.3 | 10.1 |
| 0.75 | 276.1 | 16.9 |
| 1.00 | 299.4 | 22.0 |
| 1.50 | 329.5 | 34.1 |
| 2.00 | 320.4 | 43.5 |
| 2.50 | 358.3 | 62.0 |
| 3.00 | 307.1 | 72.0 |
| 4.00 | 303.5 | 87.3 |
| 5.00 | 250.8 | 84.4 |
| 6.00 | 264.8 | 94.6 |
| 7.00 | 206.0 | 84.3 |
| 8.00 | 200.5 | 83.8 |
| 10.00 | 137.5 | 68.5 |
| 12.00 | 111.3 | 67.0 |
| 24.00 | 22.7 | 20.1 |
| 36.00 | 7.0 | 6.5 |
| 48.00 | 4.2 | 3.1 |

TABLE 22
Mean Plasma Maximum Concentration and Area Under the Curve

| Parameters | (+) Tramadol CR | (−) Tramadol IR |
|---|---|---|
| Cmax (ng/ml) | 103.4 | 398.3 |
| AUC48 hours (ng hr/ml) | 1611.6 | 3701.8 |

Based on these data, the delivery system presented herein based on a bi-layer tablet design can deliver two or more pharmaceutically active materials, each one of the active agents released at different rates in humans after oral administration.

What is claimed is:

1. A pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation, and wherein when measured by the USP type II dissolution method, the in vitro dissolution rates for the CR formulation and the IR formulation are:

| Time (hours) | % CR Release | % IR Release |
|---|---|---|
| 0 | 0% | 0% |
| 0.3 | 0-60% | 20-100% |
| 0.5 | 0-65% | 20-100% |
| 1.0 | 5-70% | 25-100% |
| 2.0 | 5-75% | 25-100% |
| 4.0 | 10-80% | 30-100% |
| 6.0 | 10-100% | 30-100% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

2. The pharmaceutical composition of claim 1, wherein the composition is a bi-layered tablet.

3. The pharmaceutical composition of claim 1, wherein the composition is formulated to provide appropriate administration to a patient without the undesirable known side effects attributed to one or the other enantiomer.

4. The composition of claim 1, wherein the CR formulation further comprises TIMERx™-N and one chiral compound enantiomer such that a gum to drug ratio of between about 1:3 to 3:1, respectively, is established.

5. The composition of claim 1, wherein the CR formulation further comprises TIMERx™-O and one chiral compound enantiomer such that a gum to drug ratio of between about 1:3 to 3:1, respectively, is established.

6. The composition of claim 1, wherein the (+) chiral compound enantiomer and the (−) chiral compound enantiomer are present in the composition at different mass quantities.

7. The composition if claim 1, wherein the (+) chiral compound enantiomer and the (−) chiral compound enantiomer are present in the composition at a percent ratio selected from the following table:

| (+) Enantiomer | (−) Enantiomer |
|---|---|
| 2 | 1 |
| 3 | 1 |
| 4 | 1 |
| 5 | 1 |
| 10 | 1 |
| 1 | 2 |
| 1 | 3 |
| 1 | 4 |
| 1 | 5 |
| 1 | 10 |

8. The composition of claim 1, wherein about 90% of the (+) chiral compound enantiomer the (−) chiral compound enantiomer are released within about 12 hours of administration.

9. a pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formation, and wherein when administered to a patient, the pharmaceutical composition provides the following percent of maximum (+) and (−) chiral compound enantiomer plasma concentrations:

| Time (hours) | (+) Enantiomer | (−) Enantiomer |
|---|---|---|
| 0 | 0% | 0% |
| 0.3 | 0-60% | 0-100% |
| 0.5 | 0-65% | 0-100% |
| 1.0 | 5-70% | 25-100% |
| 2.0 | 5-75% | 25-100% |
| 4.0 | 10-80% | 30-100% |
| 6.0 | 20-100% | 30-100% |
| 8.0 | 20-100% | 20-100% |
| 10.0 | 20-100% | 20-100% |
| 12.0 | 10-100% | 0-90% |
| 18.0 | 0-80% | 0-80% |
| 24.0 | 0-80% | 0-80%. |

10. A pharmaceutical composition for oral delivery administration comprising a pharmaceutically efficacious chiral compound, or a pharmaceutically acceptable salt thereof, wherein the pharmaceutically efficacious chiral compound, or a pharmaceutically acceptable salt thereof, comprises the (+) chiral compound enantiomer, or a pharmaceutically acceptable salt thereof, and the (−) chiral compound enantiomer, or a pharmaceutically acceptable salt thereof, each chiral compound enantiomer formulated separately, either as an immediate release (IR) formulation or as a controlled release (CR) formulation, wherein when administered to a patient, the pharmaceutical composition provides the following percent of maximum (+) and (−) chiral drug enantiomer plasma concentrations:

| Time (hours) | (+) Enantiomer | (−) Enantiomer |
|---|---|---|
| 0 | 0% | 0% |
| 0.3 | 0-40% | 0-100% |
| 0.5 | 0-45% | 0-100% |
| 1.0 | 5-50% | 25-100% |
| 2.0 | 5-55% | 25-100% |
| 4.0 | 10-80% | 30-100% |
| 6.0 | 20-100% | 30-100% |
| 8.0 | 20-100% | 20-100% |
| 10.0 | 10-100% | 20-100% |
| 12.0 | 0-80% | 10-90% |
| 18.0 | 0-80% | 0-80% |
| 24.0 | 0-80% | 0-80%. |

11. A pharmaceutical composition comprising tramadol, or a pharmaceutically acceptable salt thereof, wherein the tramadol, or a pharmaceutically acceptable salt thereof, is a combination of the two (+)and (−)tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration, and wherein when measured by the USP type II dissolution method, the in vitro dissolution rates for the CR formulation and the IR formulation are:

| | % (+) Tramadol | % (−) Tramadol |
|---|---|---|
| Time (hours) | Enantiomer Release | Enantiomer Release |
| 0 | 0% | 0% |
| 0.3 | 0-60% | 20-100% |
| 0.5 | 0-65% | 20-100% |
| 1.0 | 5-70% | 25-100% |
| 2.0 | 5-75% | 25-100% |
| 4.0 | 10-80% | 30-100% |
| 6.0 | 10-100% | 30-100% |
| 8.0 | 20-100% | 40-100% |
| 10.0 | 25-100% | 45-100% |
| 12.0 | 25-100% | 45-100% |
| 18.0 | 35-100% | 50-100% |
| 24.0 | 35-100% | 50-100%. |

12. The pharmaceutical composition of claim 11, wherein the composition is a bi-layered tablet for oral delivery.

13. The pharmaceutical composition of claim 11, wherein the composition is formulated to provide appropriate administration to a patient for the treatment of pain without the undesirable known side effects.

14. The composition of claim 11, wherein the CR formulation further comprises TIMERx™-N and one chiral compound enantiomer such that a gum to drug ratio of between about 1:3 to 3:1, respectively, is established.

15. The composition of claim 11, wherein the CR formulation further comprises TIMERx™-O and one chiral compound enantiomer such that a gum to drug ratio of between about 1:3 to 3:1, respectively, is established.

16. The composition of claim 11, wherein about 90% of the (+) tramadol enantiomer and about 90% of the (−) tramadol enantiomer are released within about 12 hours of administration.

17. The composition of claim 11, wherein the (+) tramadol enantiomer and the (−) tramadol enantiomer are present in the composition at a percent ratio of 3: 1, respectively.

18. The composition of claim 11, wherein the (+) tramadol enantiomer and the (−) tramadol enantiomer are present in the composition at a percent ratio of 2: 1, respectively.

19 A pharmaceutical composition for oral delivery administration comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration, and wherein when administered to a patient, the pharmaceutical composition provides the following percent of maximum plasma concentrations for the (+) and (−) tramadol enantiomers:

| Time (hours) | (+) Enantiomer | (−) Enantiomer |
|---|---|---|
| 0 | 0% | 0% |
| 0.3 | 0-60% | 0-100% |
| 0.5 | 0-65% | 0-100% |
| 1.0 | 5-70% | 25-100% |
| 2.0 | 5-75% | 25-100% |
| 4.0 | 10-80% | 30-100% |
| 6.0 | 20-100% | 30-100% |
| 8.0 | 20-100% | 20-100% |
| 10.0 | 20-100% | 20-100% |
| 12.0 | 10-100% | 0-90% |
| 18.0 | 0-80% | 0-80% |
| 24.0 | 0-80% | 0-80%. |

20. A pharmaceutical composition for oral delivery administration comprising tramadol, or a pharmaceutically acceptable salt thereof, wherein the tramadol, or a pharmaceutically acceptable salt thereof, is a combination of the two (+) and (−) tramadol enantiomers comprising (+) tramadol enantiomer, or a pharmaceutically acceptable salt thereof, in a controlled release (CR) formulation and the (−) tramadol enantiomer, or a pharmaceutically acceptable salt thereof, in an immediate release (IR) formulation for oral delivery administration, and wherein administered to a patient, the pharmaceutical composition provides the following percent of maximum plasma concentrations for the (+) and (−) tramadol enantiomers:

| Time (hours) | (+) Enantiomer | (−) Enantiomer |
|---|---|---|
| 0 | 0% | 0% |
| 0.3 | 0-40% | 0-100% |
| 0.5 | 0-45% | 0-100% |
| 1.0 | 5-50% | 25-100% |
| 2.0 | 5-55% | 25-100% |
| 4.0 | 10-80% | 30-100% |
| 6.0 | 20-100% | 30-100% |
| 8.0 | 20-100% | 20-100% |
| 10.0 | 10-100% | 20-100% |
| 12.0 | 0-80% | 10-90% |
| 18.0 | 0-80% | 0-80% |
| 24.0 | 0-80% | 0-80%. |

21. The composition of claim 12, wherein the bi-layer tablet consists of the following:
(a) a controlled release formulation consisting of about:

| Ingredients | A | | (%) |
|---|---|---|---|
| 1. (+) Tramadol HCl | 50 | mg | 5.4 |
| 2. TIMERx ™-N | 350 | mg | 37.7 |
| 3. Proslov | 150 | mg | 16.2 |
| 4. Magnesium Stearate | 5.5 | mg | 0.6 |
| Total | 555.5 | mg | 59.9 |

(b) an immediate release formulation consisting of about:

| Ingredients | A | | (%) |
|---|---|---|---|
| 1. (−) Tramadol HCl | 150 | mg | 16.2 |
| 2. Prosolv | 100 | mg | 10.8 |
| 3. Lactose Fast-Flow | 100 | mg | 10.8 |
| 4. Explotab | 20 | mg | 2.2 |
| 5. Magnesium Stearate | 3 | mg | 0.3 |
| Total | 373 | mg | 40.3 |

22. The pharmaceutical composition of claim 1 or claim 9 or claim 10 or claim 11 or claim 19 or claim 20, wherein the weight/weight percentage of TIMERx -N in the formulation is 38%.

23. The pharmaceutical composition of claim 1 or claim 9 or claim 10 or claim 11 or claim 19 or claim 20, wherein the weight/weight percentage of TIMERx™-O in the formulation is 38%.
